(19) United States
(12) Patent Application Publication
(10) Pub. No.: US 2009/0182137 A1
(43) Pub. Date: Jul. 16, 2009

(54) Vesicle for separating substances from liquid media

(76) Inventors: Ulrich Schwaneberg, Ritterhude (DE); Mathias Winterhalter, Bremen (DE); Wolfgang Meier, Therwill (CH)

Correspondence Address: SUGHRUE MION, PLLC, 2100 PENNSYLVANIA AVENUE, N.W., SUITE 800, WASHINGTON, DC, 20037, US

(21) Appl. No.: 10/588,205

(22) PCT Filed: Feb. 2, 2005
(86) PCT No.: PCT/EP05/01039

§ 371 Date: Apr. 24, 2008

(30) Foreign Application Priority Data
Feb. 2, 2004 (EP) 04002214.7

(51) Int. Cl. C07H 1/00; C12N 1/00

(52) U.S. Cl. 536/25.41; 435/317.1

(57) ABSTRACT

The invention relates to methods of separating substances from liquid media and to separating agents suitable therefor.

The invention relates to methods of separating substances from liquid media and to separating agents suitable therefor.

A number of methods and separating agents are known for separating substances from liquid media. Adsorptive methods, such as adsorption chromatography, ion-exchange chromatography and the like, have acquired particular importance in the field of biochemistry. In adsorptive methods, the substance to be separated binds to a carrier material, in most cases a solid phase, and is separated from the liquid medium together with the carrier material. Carrier materials having a large specific surface area, in particular glass beads and polymer beads, are conventionally used. In order to increase the surface area, the carrier materials can additionally be porous, so that surfaces for binding the substance to be separated are made available even on the inside of the carrier material. In order to ensure that the diffusion of the substance to be separated to the inside of a porous carrier is as good as possible, and accordingly to permit a good adsorption efficiency, a large pore cross-section is chosen, so that transportation of the substance—in particular by diffusion—into the inside of the carrier is not or is only negligibly impeded. The average pore diameters are therefore conventionally a multiple of the size of the substance to be separated.

With conventional separating methods, however, a specific substance or a specific group of substances can be separated from a mixture of several chemically similar substances only with a large outlay. “Beads” provided with antibodies or their binding sites are frequently used for that purpose. They are complex, however, and relatively expensive to prepare, because the corresponding antibodies first have to be produced. In addition, they are suitable to only a limited extent for distinguishing nucleic acids from one another and separating them selectively from a liquid medium.

An object of the present invention was, therefore, to provide separating agents for separating a substance from a liquid medium. The separating agents should be as simple as possible to prepare, should permit the selective separation of as wide a variety of different substances as possible, should be stable in a large number of conventional liquid media, such as cell culture media and cell extracts, and they should be able to separate as large a proportion as possible of the substance, present in the medium, that is to be separated. Corresponding separating methods and uses of the separating agents are also to be provided.

The object is achieved according to one aspect by vesicles
having a membrane containing amphiphilic molecules,
a pore-forming pore unit contained in the membrane, in order to allow access to the vesicle interior,
   wherein the vesicle contains, in the vesicle interior, a binding substance for binding the substance to be bound, and wherein the binding substance is substantially unable to diffuse through the pore formed by the pore unit.

By the incorporation or provision of a binding substance enclosed in the vesicle interior it becomes possible for the first time permanently to bind relatively large amounts in the interior of the vesicle solely by chemical binding or chemical interactions. This enables, for example, nucleic acids or other charged macromolecules to be retained in the interior of such a vesicle for a prolonged period, even with open pores. Accordingly, the vesicle according to the invention is suitable in particular as a separating agent for separating a substance from a liquid medium by selecting the binding substance in such a manner that the substance to be separated is bound by the binding substance, so that the bound substance can be separated from the liquid medium with the vesicle itself, for example by filtration or sedimentation/centrifugation. Unlike conventional porous separating agents, the vesicles according to the invention additionally have from the outset a specificity for those substances that are able to pass through the pore formed by the pore unit and have an affinity for the binding substance. The vesicles according to the invention are therefore able to separate, for example, similar substances, such as PCR primers or double-stranded PCR products, from larger nucleic acids such as, for example, plasmid DNA.

The membrane preferably contains as the amphiphilic molecule an amphiphilic copolymer having a hydrophilic and a hydrophobic portion. It is particularly preferred for the membrane of the vesicle according to the invention to be formed from amphiphilic block copolymer molecules, and it is very particularly preferred for the membrane to be formed from a single type of amphiphilic block copolymer molecules.

Vesicles of amphiphilic block copolymers have already been described previously as bioreactors (Nardin et al., Eur. Phys. J. E 4, (2001), 403-410, “Amphiphilic block copolymer nanocontainers as bioreactors”; as well as WO 01/32146). Those vesicles are vesicles or micelles of aggregating block copolymers having at least one hydrophilic and one hydrophobic portion. The resulting vesicles/micelles are very stable to dilution, can possess a monodisperse size distribution and have a very low dynamics, so that their half-life can be up to several hours. The described bioreactors are not suitable for the concentration and separation of substances, however. In particular, substances having a high molecular weight of, for example, more than 1000 Da are unable to diffuse into the bioreactor. Furthermore, the bioreactors do not possess any means for taking up substances into their interior in a higher concentration than in the solution. In addition, the substances taken up into the bioreactor are reacted to form products that leave the bioreactor again according to a concentration gradient.

Also known are vesicles in which a polymer skeleton is synthesised in the interior of the membrane of a liposome by polymerisation (Hotz and Meier, Langmuir 1998, 14, 1031-1036, “Vesicle-Templated Polymer Hollow Spheres”). These vesicles are able to form stable hollow spheres even in the dry state after removal of the surface-active substances used for the polymerisation.

For the preparation and processing of the vesicles and, in particular, of the amphiphilic copolymers contained in the vesicle membrane, the person skilled in the art will make reference in particular to the above-mentioned publications of C. Nardin et al. (Eur. Phys. J. E 4, (2001), 403-410) as well as international patent application WO 01/32146. The embodiments described therein of corresponding vesicle membranes and of the amphiphilic copolymers contained therein are also particularly preferred according to the invention.

It is particularly preferred for the amphiphilic copolymer to be a segmented copolymer having at least one hydrophilic portion A and at least one hydrophobic portion B, the segmented copolymers being able to self-assemble to form vesicles. It is also possible for the amphiphilic copolymer to contain more than one hydrophilic portion and more than one hydrophobic portion; in particular, the copolymer can have an ABA structure with two hydrophilic portions and a hydrophobic portion arranged between them. Block copolymers are preferred for the formation of vesicles according to the invention, which block copolymers can in particular be linear. Instead of or in addition to linear block copolymers it is possible, however, also to use graft copolymers or comb structures which possess both (at least) one hydrophilic portion and (at least) one hydrophobic portion.

If an ABA copolymer is used to form a vesicle according to the invention, then a vesicle having a hydrophilic inner and outer layer and a hydrophobic intermediate layer is formed. If a BAB copolymer is used, then a vesicle having a hydrophobic inner and outer layer and a hydrophilic intermediate layer is formed. Further preferred copolymers and their possible uses to form vesicles can be found in particular in WO 01/32146.

In a further preferred embodiment of the vesicles according to the invention, the membrane contains a plurality of amphiphilic copolymers, it being possible for the amphiphilic copolymers to be bonded together covalently. The copolymers can be bonded together in particular by polymerisation. Examples of such copolymers that are preferred according to the invention, and of vesicle membranes formed therefrom, are likewise to be found in WO 01/32146 or the mentioned publication of C. Nardin et al.

Accordingly, the membrane particularly preferably contains a poly-(2-methyloxazoline) block-poly(dimethylsiloxane) block-poly-(2-methyloxazoline) triblock copolymer (PMOXA-PDMS-PMOXA), preferably having one or more polymerisable groups at both chain ends, as also described in Example 1 of WO 01/32146. This copolymer is particularly suitable for the formation of stable vesicles and facilitates the incorporation of a pore-forming pore unit into the membrane.

It is not necessary for the membrane of the vesicle according to the invention to be composed solely of amphiphilic copolymers of a single type. In particular, the membrane can also contain further types of amphiphilic copolymers in addition to PMOXA-PDMS-PMOXA triblock copolymers.

The binding substance binds to the substance to be bound via a chemical bond, that is to say with the formation of a primary valence bond, for example a covalent bond or an ionic bond, a secondary valence bond, for example a Van der Waals interaction and/or a hydrogen bridge bond, and/or by a hydrophilic or hydrophobic interaction. It is particularly preferred for the binding substance to be equipped to form one or more hydrogen bridges, ionic bonds and/or for hydrophobic interaction with the substance to be bound. These types of bond permit specific binding to numerous substances that are of relevance in biochemical practice; they additionally allow the bound substance to be detached from the binding substance again by simple means. In particularly preferred embodiments, the vesicles according to the invention are equipped to concentrate the substance to be separated in their interior in comparison with a surrounding solution.

The binding substance contained in the vesicle interior is preferably a polyelectrolyte. It is preferred to use a poly acid as the binding substance when the vesicle interior is to bind one or more positively charged substances. Examples of suitable poly acids are polyphosphoric acid, polyvinylsulfuric acid, polyvinylsulfonic acid, polyvinylphosphonic acid and polyacrylic acid. If, however, the vesicle interior is to bind one or more negatively charged substances, then it is preferably provided with a poly base, for example polyethyleneimine, polyvinylamine, polyvinylpyridine and/or polylysin.

In addition or as an alternative thereto, the binding substance can also be an oligo acid or an oligo base of from 10 to 100 acidic or basic fundamental units. The person skilled in the art can select appropriate oligo and poly acids or bases in particular according to whether they are able to diffuse through a pore of the pore-forming unit, it being advantageous to select those binding substances that are capable of such diffusion to only a very small degree.

In addition or as an alternative to the use of poly acids or oligo acids or bases, an affinity for the substance to be bound can be formed in the vesicle interior also by a reaction which takes place after formation of the vesicle with joining and/or dissolution of a covalent bond of units contained in the vesicle membrane.

In addition, it is particularly preferred for the vesicle interior to be substantially hollow. This allows substantially the whole of the volume enclosed by the vesicle membrane to be used for the binding of the substance to be bound. In addition, a bound substance is protected by the vesicle membrane; for example, proteases from a surrounding medium are no longer able to pass into the vesicle interior.

It is likewise particularly preferred for the vesicle membrane to be free of the binding substance on its outside. The substance to be bound is then bound mainly by the binding substance present in the vesicle interior; this allows the particularly good specificity achievable by the pore unit to be utilised for distinguishing between similar substances (for example distinguishing short PCR products from large plasmids).

The pore unit is preferably equipped to regulate the permeation of substances (preferably the substance to be bound in particular) into the vesicle interior.

Particular preference is given to those vesicles whose pore unit contains a protein or a protein part selected from the group consisting of
a) a pore-forming transmembrane protein,
b) a pore-forming transmembrane protein having an alpha-helical transmembrane structure, in particular alamethicin, melittin, magainin, dermaseptin,
c) a pore-forming transmembrane protein having a β-barrel transmembrane structure, in particular Rhodobacter capsulatus porin, Rhodopseudomonas blastica porin, Omp, ScrY, FepA, PhoE and, in particular for substances having a molecular weight≦1000 Da, OmpF, LamB, OmpK36 and for substances having a molecular weight>1000 Da FhuA, TOlC, maltoporin and alpha-haemolysin,
d) a transmembrane structure of a pore-forming transmembrane protein, and
e) a protein having a structure that is structurally homologous to a pore-forming transmembrane structure of one of the proteins according to a), b), c) and/or d).

The person skilled in the art will find more detailed information on the proteins listed above in the corresponding publications of Alberts et al., Molecular Biology of the Cell, Garland Publishing, Inc., 3rd Ed., 1994, p. 485-498 including further references; Biggin P C and Sansom M S, Biophys. Chem. 1999, 76(3): 161-183; Pawlak et al., Protein Sci. 1994, 3(10): 1788-1805, Template assembled melittin: structural and functional characterization of a designed, synthetic channel-forming protein; Sakai N and Matile S, Chem Commun (Camb.) 2003, (20): 2514-2523, Synthetic multifunctional pores: lessons from rigid-rod beta barrels; Weiss et al., FEBS Lett. 280 (1991), The structure of porin from Rhodobacter capsulatus at 1.8 Å resolution; Kreusch et al., Protein Sci. 3 (1994): 58-63, Structure of the membrane channel porin from Rhodopseudomonas blastica at 2.0 Å resolution; Cowan et al., Nature 358 (1992): 727-733, Crystal structures explain functional properties of two E. coli porins; Dutzler et al., Struct. Fold. Des. 7 (1999): 425-434, Crystal structure and functional characterization of OmpK36, the osmoporin of Klebsiella pneumoniae; Zeth et al., Structure Fold Des. 8 (2000): 981-992, Crystal structure of Omp32, the anion-selective porin from Comamonas acidovorans, in complex with a periplasmic peptide at 2.1 Å resolution; Schirmer et al., Science 267 (1995): 512-514, Structural basis for sugar translocation through maltoporin channels at 3.1 Å resolution; Danelon et al., J Biol. Chem. 2003, 278(37): 35542-35551, Probing the orientation of reconstituted maltoporin channels at the single-protein level; Forst et al., Nat. Struct. Biol. 5 (1998): 37-46, Structure of the sucrose-specific porin ScrY from Salmonella typhimurium and its complex with sucrose; Buchanan et al., Nat. Struct. Biol. 6 (1999): 56-63, Crystal structure of the outer membrane active transporter FepA from Escherichia coli; Fergusson et al., Science 282 (1998): 2215-2220, Siderophore-mediated iron transport: crystal structure of FhuA with bound lipopolysaccharide; Locher et al., Cell 95 (1998): 771-778, Transmembrane signalling across the ligand-gated FhuA receptor: crystal structures of free and ferrichrome-bound states reveal allosteric changes; Koronakis et al. and Song et al. as indicated above.

A structure is regarded as being structurally homologous to a starting structure when, like the starting structure, it permits the formation of a pore in a vesicle membrane.

In preferred embodiments of the invention, the permeation of substances into the vesicle interior is regulated by the pore unit in particular in that the pore unit allows substances having a preselected minimum and/or maximum size and/or a preselected minimum and/or maximum charge density to pass through the vesicle membrane. The use of a pore unit makes it possible, in an advantageously simple manner, to establish a constant, preselectable pore size and to provide the pore properties necessary for otherwise regulating the permeation (in particular charge distributions) in a constant and preselectable manner and to change them in a targeted manner by means of molecular biological methods (e.g. mutagenesis) in order, for example, to change selectivities and transport properties.

In preferred vesicles, the pore unit contains a protein or a protein part that contains a β-barrel structure. A β-barrel structure occurs very frequently in integral membrane proteins. In particular, it is a frequent feature of proteins that are involved in the transport of substances through the outer bacterial membrane. Porins or their respective β-barrel domain or their β-barrel structure have been particularly well characterised and are preferred according to the invention as the pore unit.

The pore unit does not have to consist solely of a protein but can also be formed from two or more protein units which cooperate to form a pore. The partial proteins in the pore unit preferably form a wall which penetrates the membrane and, seen overall, has a β-barrel structure. Examples of such pore units, which are likewise preferred, whose β-barrel structure arises from the cooperation of several protein units are the trimeric TolC protein from E coli and the heptameric staphylococcal haemolysin toxin (see Koronakis et al.; Nature 405 (2000): 914-919; Crystal structure of the bacterial membrane protein TolC central to multidrug efflux and protein export, or Song et al., Science 274 (1996): 1859-1866, Structure of staphylococcal α-hemolysin, a heptameric transmembrane pore). Any mention of a β-barrel structure within the scope of the present text always includes a portion of a protein that, when it cooperates with further suitable portions of optional further proteins, forms in a membrane of a vesicle according to the invention a pore having a β-barrel structure.

A particularly preferred pore unit is the FhuA channel-forming protein or a protein having the β-barrel domain thereof or having a structure that is structurally homologous thereto. This protein has high temperature stability (up to about 60° C.), it permits the transport of phage DNA into the vesicle interior, and it is expressable in conventional host cells, for example in E. coli. The protein has a substantially elliptical pore diameter of from 4.6 nm to 3.9 nm. The β-barrel domain is formed by 22 antiparallel folded sheets.

An example of a protein having the β-barrel structure of FhuA is a FhuA derivative obtained by removal of amino acids 5 to 160 from FhuA (see Braun et al., Eur. J. Biochem. 269 (2002): 4948-4959, Diffusion through channel derivatives of the Escherichia coli FhuA transport protein). This FhuA derivative is particularly preferred as the pore unit. It forms a pore having an inside diameter which is about 1 nm wide at the narrowest point. By suitable amino acid substitution, the pore width can be narrowed or widened or the pore can be provided with charges on the inside of the pore.

It is further preferred for the pore unit to have an inside pore diameter having a width of more than 0.1 nm, preferably at least 1 nm and particularly preferably at least 3.5 nm. Pore units having corresponding pore diameters can be produced particularly simply by means of β-barrel structures of known proteins, as just shown with reference to the particularly preferred example of the FhuA protein. In addition, corresponding pore units allow not only small atoms and molecules but also larger molecules, in particular nucleic acids such as DNA and RNA, to pass into the interior of the vesicles according to the invention. Corresponding pore units are therefore preferably used according to the invention to separate a nucleic acid, in particular a DNA and/or a RNA.

It is also preferred according to the invention for the pore unit to have an inside pore diameter having a width of not more than 10 nm, preferably not more than 8 nm and particularly preferably not more than 5 nm. In an advantageously simple manner, the pore unit thus allows the vesicles according to the invention to be used in the manner of a screen and the substances that are able to pass into the vesicle interior to be selected on the basis of their width.

If nucleic acids, in particular single- and/or double-stranded DNA or RNA, are to be taken up in the vesicle according to the invention, then the pore diameter is preferably at least 1 nm and not more than 5 nm.

Particular preference is given according to the invention also to those vesicles that have a positively charged oligomer and/or polymer in the vesicle interior. Examples of corresponding oligomers and polymers have already been described hereinbefore. Particular preference is given according to the invention to those vesicles that contain polylysin in the vesicle interior. Using corresponding oligomers and polymers, in particular containing polylysin, vesicles according to the invention which are suitable in particular for taking up and binding nucleic acids, in particular single- and double-stranded DNA or RNA, in the vesicle interior can be formed very simply. Advantageously, a sufficiently high concentration of the positively charged oligomer and/or polymer (in particular of polylysin) is provided in the vesicle interior to ensure that the nucleic acid (in particular single- and/or double-stranded DNA or RNA) to be taken up in the vesicle is retained securely.

Accordingly, it is further preferred to use vesicles according to the invention for binding a charged substance. On account of their particular structure described above, vesicles according to the invention are particularly suitable for taking up one or more electrically charged substances in their interior. By using suitable substances, for example substances that bring about a potential difference, the vesicles according to the invention can be used particularly successfully for retaining and binding one or more electrically charged substances in their interior. By suitably selecting a pore former, the vesicles according to the invention can additionally be used for taking up, from a mixture of different electrically charged substances, only those substances having a preselected maximum size, and optionally binding them. Particularly suitable pore formers have already been described hereinbefore; particular preference is given to a FhuA protein or a protein having the β-barrel domain of FhuA as the pore former in a vesicle according to the invention for taking up and optionally binding an electrically charged substance.

It is likewise preferred to select as the pore unit a protein or partial protein of one of the types described above which has a positive or negative net charge inside the pore. Such a pore allows passage through the pore to be facilitated for those substances that possess an opposite charge and to be made more difficult for those substances that possess the same charge. In particular, a charged binding substance can in this manner be retained in the vesicle interior even if, on account of its size, it would otherwise be able to diffuse through the pore formed by the pore former.

In addition, it is preferred for the pore unit to form an enantioselective pore. An example of an enantioselective pore unit is maltoporin (Danelon et al., see above). In addition, by the incorporation of suitable amino acids, transmembrane proteins or their transmembrane structures which were previously not enantioselective can be rendered enantioselective. Furthermore, it is to be expected that further enantioselective transmembrane proteins will be found. The use of an enantioselective transmembrane protein or of an enantiomeric transmembrane structure in a pore unit (or as the pore unit) of a vesicle having a membrane containing amphiphilic copolymers is likewise preferred.

It is also particularly preferred for the substance to be taken up and/or bound in the vesicle to be a nucleic acid. The vesicles according to the invention, with suitable selection of the pore unit, allow nucleic acids to be separated from liquid mixtures, in particular of proteins, simply and highly efficiently.

In preferred embodiments, the charges in the interior of the vesicles according to the invention can be modulated and adapted to many different problems in particular via the pH value, via the salt concentration or the number of ionic groups in the vesicles.

The substances taken up and/or bound in the vesicles according to the invention can be removed therefrom again a) by destroying the vesicles mechanically, for example by applying a shear force, b) by dissolving the vesicles, for example in ethanol, and/or c) by adding a salt, for example NaCl.

Accordingly, it is preferred to use the vesicles according to the invention for taking up and/or for binding a nucleic acid. Such a use of the vesicles according to the invention allows a nucleic acid to be separated quickly from a mixture containing further charged substances, for example proteins. The use of the vesicles according to the invention for this purpose also allows nucleic acids to be obtained from corresponding mixtures in high yields. In addition, in particularly preferred embodiments, no agarose is required to obtain the nucleic acids, and finally, only small amounts of waste are formed in the case of the use according to the invention, it being possible largely to dispense with carcinogenic reagents.

Preference is therefore given also to a method of separating a nucleic acid from a nucleic-acid-containing solution, which comprises bringing the nucleic acid to be separated into contact with a vesicle according to the invention as described above. It is advantageous to use vesicles according to the invention that have a positively charged oligomer and/or polymer in the vesicle interior. It is particularly preferred to use vesicles containing polylysin. In preferred embodiments of the method, the pore size of the vesicles according to the invention that are used is from 1 to 5 nm; a particularly preferred pore unit is a FhuA derivative which, compared with the natural FhuA protein, lacks amino acids 5 to 160.

The method according to the invention, in particular using the preferred vesicles according to the invention just described, including the particularly preferred pore units, also allows nucleic acids of different sizes to be separated from one another. For example, with a suitably selected pore unit (in particular a pore unit having a β-barrel structure which is structurally homologous to that of FhuA), large nucleic acids such as plasmid DNA are unable to pass into the interior of the vesicles according to the invention because of their width, while shorter, non-circular and, in particular, single-stranded nucleic acids are able to pass. Owing to this screening action of the pore unit, the method according to the invention additionally allows nucleic acids to be separated from proteins quickly and with a high yield.

By appropriately adjusting the salt concentration and the pH value, permanent binding of short single-stranded nucleic acids in the interior of the vesicles according to the invention can additionally be prevented.

A particularly preferred method of separating a nucleic acid comprises the steps:
a) bringing a solution containing the nucleic acid to be separated into contact with a vesicle according to the invention as described above, which vesicle preferably contains polylysin and the membrane of which contains a pore former containing the β-barrel domain of a FhuA protein;
b) allowing the nucleic acid to be separated to penetrate into the interior of the vesicle; this can take place especially at room temperature;
c) separating the vesicles according to the invention from the solution originally containing the nucleic acid to be separated; this can take place, for example, by centrifugation and/or filtration;
d) adjusting the salt concentration in order to separate from the vesicle according to the invention nucleic acids that have a smaller size than the nucleic acid to be separated (for example PCR primers), and separating off the nucleic acids so freed; and
e) freeing the nucleic acids which have been separated, by
   a. destroying the vesicle by applying a shear force,
   b. dissolving the vesicle by adding ethanol and/or
   c. by addition of high NaCl concentrations and subsequently removing the salts over a conventional chromatography column.

Such a method can be completed within 30 to 45 minutes and is therefore unusually quick compared with conventional methods.

The use of a pore unit of one of the above-described types to form a pore in a vesicle whose membrane contains an amphiphilic copolymer as described above is likewise preferred according to the invention.

Preferred embodiments of the invention are explained further hereinbelow with reference to the Figures and the accompanying Examples. The Figures show:

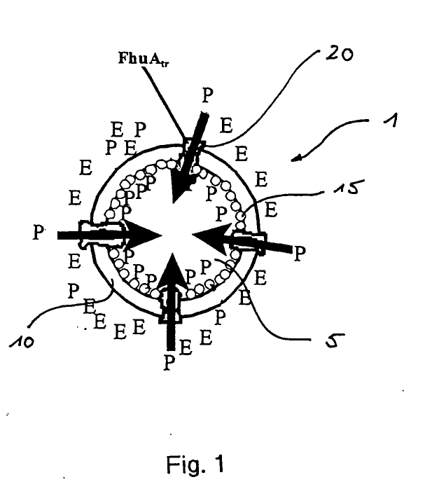

FIG. 1 a representation, in diagrammatic form, of the structure of a vesicle according to the invention;

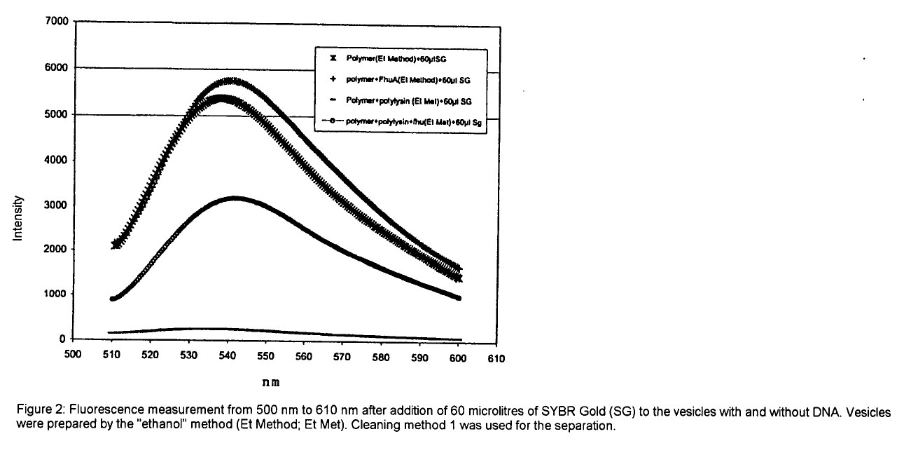

FIG. 2 fluorescence measurement from 500 nm to 610 nm after addition of 60 μl of SYBR Gold to the vesicles with and without DNA, see Example 3, Method 1;

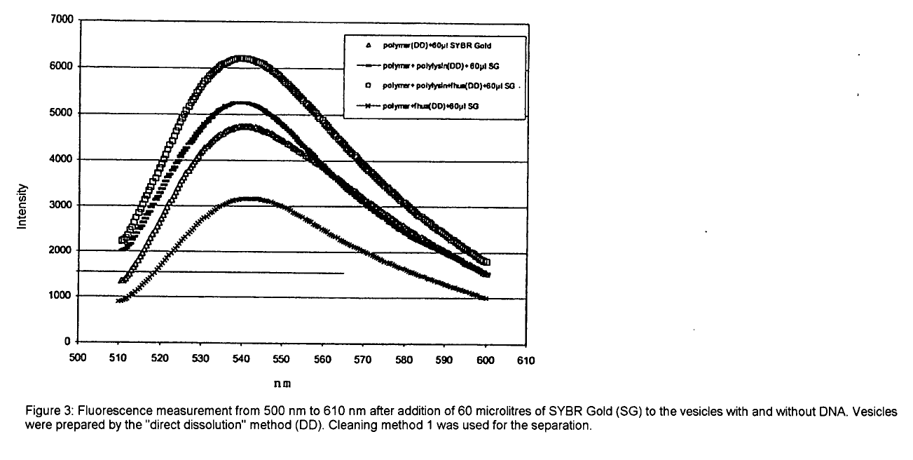

FIG. 3 fluoresence measurement from 500 nm to 610 nm after addition of 60 μl of SYBR Gold to the vesicles with and without DNA, see Example 3, Method 1;

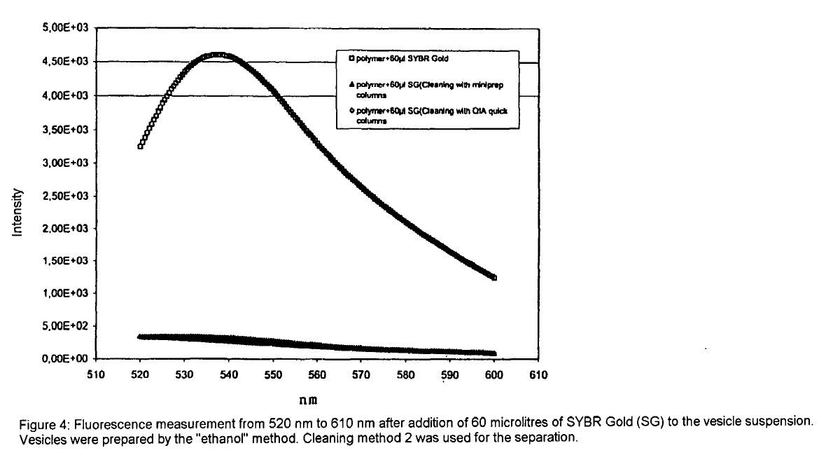

FIG. 4 fluoresence measurement from 500 nm to 610 nm after addition of 60 μl of SYBR Gold to the vesicles with and without DNA, see Example 3, Method 2;

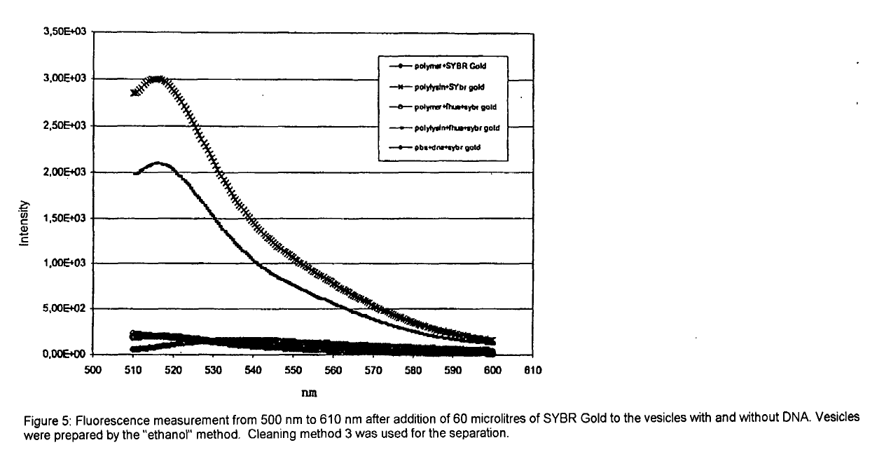

FIG. 5 fluoresence measurement from 500 nm to 610 nm after addition of 60 μl of SYBR Gold to the vesicles with and without DNA, see Example 3, Method 3; and

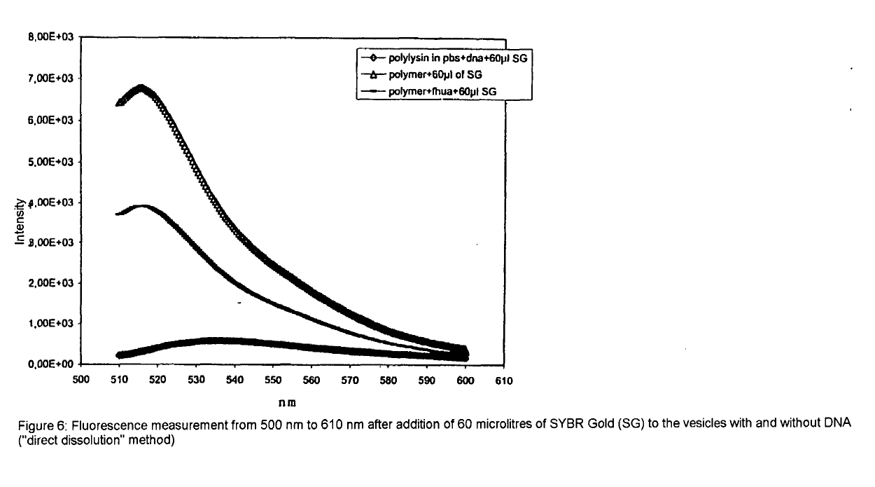

FIG. 6 fluoresence measurement from 500 nm to 610 nm after addition of 60 μl of SYBR Gold to the vesicles with and without DNA, direct dissolution.

FIG. 1 shows, in diagrammatic form, the structure and mode of action of a vesicle according to the invention for separating a charged substance. The vesicle 1 has a membrane 10 of PMOXA-PDMS-PMOXA triblock copolymer. The vesicle 1 is substantially spherical and has a vesicle interior 5. The vesicle interior 5 is free of solids.

The membrane 10 contains four pore units 20, each of which forms a channel allowing a substance (arrows) to enter the vesicle interior 5. The pore units 20 are FhuAₜᵣ proteins.

On the inside 12 of the membrane 10 there is arranged a polyelectrolyte 15. The polyelectrolyte 15 has a charge suitable for binding the charged substance. If the substance to be bound is a nucleic acid, then the polyelectrolyte is preferably polylysin.

The charged substance passes through a pore 20 in the membrane 10 of the vesicle 1 into the vesicle interior 5, where it binds to the polyelectrolyte 15.

In order to release the charged substance from the vesicle 1, the vesicle 1 is destroyed by application of a shear force (not shown). The vesicle 1 can also be dissolved by addition of ethanol and the bound substance can be released in that manner. A third way of releasing the bound substance is the addition of suitable ions, so that the charged substance is detached from the polyelectrolyte 15. The two last-mentioned methods avoid exposing the bound substance to high mechanical stress.

### EXAMPLE 1

### Preparation of FhuAₜᵣ

Bacterial strain and growth conditions: E. coli strain DH5α is used. The cells are cultured at 37° C. in LB medium (10 g/l tryptone, 5 g/l yeast extract, 10 g/l NaCl) with shaking at 200 rpm. Ampicillin is added to a final concentration of 100 μg/ml, and the optical density at 578 nm is monitored. When the optical density at 578 nm reaches a value of 0.9-1, anhydrotetracycline (200 μg/l cell culture) is added in order to induce the tet promoter on the plasmid contained in the bacteria. The cells are incubated for a further 3 h (3 hours).

Plasmid: The plasmid used is pFHUA, a pASK-IBA3 derivative (IBA-Labortechnik, Göttingen). The wild-type gene was obtained from Biozentrum Basel (Braun, M., Killmann, H., and Braun, V., The β-barrel domain of FhuAΔ5-160 is sufficient for TonB-dependent FhuA activities of Escherichia coli, Mol. Microbiol. (1998)). A EcoRI restriction site is incorporated into the wild-type gene at amino acid position 129 (FhuA Δ1-129) and at amino acid position 160 (FhuA Δ1-160). A XhoI restriction site was incorporated in both cases at the ends of the genes with the aid of the PCR primers SEQ ID NO 1 and SEQ ID NO 2. The amplified DNA fragment was cleaned by agarose gel electrophoresis and with QIAQuick kits (Qiagen), digested with EcoRI (New England Biolabs) and XhoI (New England Biolabs), again cleaned by agarose gel electrophoresis and ligated with the linearised pASK-IBA3 plasmid (cleaved with EcoRI and XhoI) and transformed into DH5α by standard cloning methods.

TABLE 1
PCR mixture for FhuAₜᵣ:

| Substance | Volume (μl) |
|---|---|
| ddH₂O | 83.4 |
| 10x buffer (Taq buffer) | 10 |
| DNTP (10 mM) | 2 |
| Primer SEQ ID NO 1 (20 mM) | 1.3 |
| Primer SEQ ID NO 2 (20 mM) | 1.3 |
| Plasmid with FhuA (wild-type gene) | 1 |
| Taq DNA polymerase (5 U/μl) | 1 |
| Total | 100 |

TABLE 2
PCR conditions

| Temperature (° C.) | Time (min) | Number of cycles |
|---|---|---|
| 94 | 3 | 1 |
| 94 | 1 | 31 |
| 68 | 1 | 31 |
| 72 | 3 | 31 |
| 72 | 10 | 1 |
| 8 | unlimited | 1 |

TABLE 3
Digestion of the plasmid pASK-IBA3

| Substance | Volume (μl) |
|---|---|
| ddH₂O | 68.16 |
| 10x buffer (NEB Buffer 2) | 10 |
| Plasmid pASK-IBA3 | 20 |
| EcoR I (20 U) | 0.42 |
| Xho I (20 U) | 0.42 |
| BSA | 1 |
| Total | 100 |

TABLE 4
Digestion of the PCR product

| Substance | Volume (μl) |
|---|---|
| ddH₂O | 16 |
| 10x buffer (NEB Buffer 2) | 10 |
| PCR product | 70 |
| EcoR I (20 U) | 2 |
| Xho I (20 U) | 2 |
| Total | 100 |

TABLE 5
Digestion conditions

| Temperature | Time |
|---|---|
| 37° C. (XhoI) | 4 h |
| 37° C. (EcoRI) | 4 h |

TABLE 6
Ligation of FhuAₜᵣ in pASK-IBA3 in order to obtain pFHUA

| Substance | Volume (μl) |
|---|---|
| ddH₂O | 5 |
| 10x buffer (ligase buffer) | 2.5 |
| Plasmid pASK-IBA3 | 4 |
| Insert (FhuAₜᵣ) | 12 |
| Ligase (T4 DNA ligase) | 1.5 |
| Total | 25 |

Extraction and isolation of FhuAₜᵣ: After overexpression of FhuA, the DH5α cells were harvested and centrifuged for 10 minutes at 2800 rpm, 4° C. The supernatant was discarded. 5 ml of lysis buffer (50 mM Na phosphate buffer, 100 mM NaCl, 2 mM EDTA and DNase I, pH 7.5) are added to 1 g of the cell pellet. The mixture is treated twice with a French Press. The lysate is centrifuged for 10 minutes at 7000 rpm. The supernatant is centrifuged for 40 minutes in an ultracentrifuge at 18° C., 30,000 rpm.

The pellet so obtained is dissolved in 20 ml of pre-extraction buffer 1 (20 mM Na phosphate buffer, 0.3% octyl-POE (n-octyl-oligo-oxyethylene, Alexis) and homogenised, a mixer being used first and then the detergent being added. The mixture is incubated for 50 minutes at 40° C. and then centrifuged in an ultracentrifuge as described above.

The pellet so obtained is dissolved in 20 ml of pre-extraction buffer 2 (20 mM Na phosphate buffer (pH 7.5), 0.5% octyl-POE) and homogenised, a mixer being used first and then the detergent being added. The mixture is incubated for 50 minutes at 40° C. and then centrifuged in an ultracentrifuge as described above.

20 ml of extraction buffer (20 mM Na phosphate buffer, 3% octyl-POE) are added to the resulting pellet, and incubation is carried out for 40-60 minutes at 37° C. After ultracentrifugation again as described above, FhuA is obtained in the supernatant. The sample of FhuA is analysed on a SDS gel. The FhuA is obtained by dialysis against 1 vol. % octyl-POE in Na phosphate buffer pH 7.5 and can be stored in that form.

### EXAMPLE 2

### Preparation of Vesicles According to the Invention

### EXAMPLE 2.1

### Direct Dissolution

50 mg of the ABA (PMOXA-PDMS-PMOXA) triblock copolymer (Nardin, C., Widmer, J., Winterhalter, M., and Meier, W., Amphiphilic block copolymer nanocontainers as bioreactors, Eur. Phys. J. (2001)) were dissolved in 4.95 ml of PBS (0.37 M NaCl, 27 mM KCl, 43 mM Na₂HPO₄ 8H₂O, 14 mM KH₂PO₄ 2H₂O, pH 7.3) and stirred overnight in order to form self-organised vesicles. These were extruded three times with a 0.4 μm filter (Millipore) in order to obtain vesicles of uniform size.

In order to prepare the membranes of the vesicles for taking up FhuA, Triton X-100 (final concentration 5%) is added. About 80 μl of FhuA are added to the mixture, and incubation is carried out for 3 h. Detergents can stabilise the vesicles, and the detergent was therefore separated off using biobeads (Rigaud, J. L., Paternostre, M. T., and Bluzat, A., Mechanisms of membrane protein insertion into liposomes during reconstitution procedures involving the use of detergents 2. Incorporation of the light-driven proton pump bacteriorhodopsin, Biochemistry (1988)), and incubation was carried out for 5-6 h with slow stirring. Samples are cleaned by Sepharose 4B in order to remove unreconstituted FhuA and other impurities. The vesicles are cleaned by centricon YM-30 (Millipore).

### EXAMPLE 2.2

### Ethanol Method

50 mg of the ABA (PMOXA-PDMS-PMOXA) triblock copolymer were dissolved in 250 μl of ethanol (99.8%) for several minutes in order to obtain a clear solution. The solution is added dropwise from above to 4.95 ml of PBS (0.37 M NaCl, 27 mM KCl, 43 mM Na₂HPO₄ 8H₂O, 14 mM KH₂PO₄ 2H₂O, pH 7.3). At the same time, 80 μl of FhuA solution are added slowly. The mixture is stirred for 3-4 h. Extrusion was carried out three times with a 0.4 μm filter (Millipore) in order to obtain vesicles of uniform size. Biobeads were used to separate off the detergent as in Example 2.1, and the remainder of the method proceeds as in Example 2.1.

### EXAMPLE 3

### Preparation of DNA-Binding Vesicles

50 mg of the ABA (PMOXA-PDMS-PMOXA) triblock copolymer are dissolved in 4.95 ml of polylysin solution (0.5 mg/ml of the polylysin solution; mol. wt. 15,000-30,000 Sigma in 5 ml of PBS) and treated further as described in Example 2.1 or 2.2.

After concentration by centricon YM-30 (Millipore), 50 μl of DNA (60mer, 20 μmol.) were added to the suspension, and incubation was carried out for 30 minutes at room temperature.

Three further methods were developed for separating DNA which was not bound in the vesicles according to the invention:

### Method 1:

The suspension was concentrated six times with centricon YM-30 (Millipore) and PBS and diluted again. After these repeated concentration and washing steps, 1 ml was used further as the sample and the content of DNA enclosed in vesicles was determined by addition of 60 μl of SYBR Gold (1× staining; Molecular Probes, Leiden, Netherlands) (see FIG. 2, 3).

### Method 2:

In this method, Qiagen cleaning kits (Qia Miniprep, Qiaquick) were used to separate the DNA inside the vesicles from the DNA outside the vesicles. The eluted solution with the vesicles was centrifuged (3000 g, 2 minutes) and the content of DNA enclosed in vesicles was determined by addition of 60 μl of SYBR Gold (1× staining; Molecular Probes, Leiden, Netherlands) (see FIG. 4).

### Method 3:

In order to remove unbound DNA which had not passed into the vesicles, a Sepharose 4B column was additionally used. The content of DNA enclosed in vesicles was determined by addition of 60 μl of SYBR Gold (1× staining; Molecular Probes, Leiden, Netherlands) (see FIG. 5, 6).

1. Vesicle for binding a substance,
having a membrane containing amphiphilic molecules,
a pore-forming unit contained in the membrane, in order to allow access to the vesicle interior,
characterised in that the vesicle contains, in the vesicle interior, a binding substance for binding the substance to be bound, and wherein the binding substance is substantially unable to diffuse through the pore formed by the pore unit.

2. Vesicle according to claim 1, characterised in that the binding substance is equipped to provide an ionic bond, a hydrogen bridge bond and/or a hydrophobic interaction.

3. Vesicle according to any one of the preceding claims, characterised in that the pore unit contains a protein or a protein part selected from the group consisting of
a) a transmembrane protein,
b) a transmembrane protein having an alpha-helical transmembrane structure,
c) a transmembrane protein having a β-barrel transmembrane structure,
d) a transmembrane structure of a transmembrane protein, and
e) a protein having a structure that is structurally homologous to a transmembrane structure of one of the proteins according to a), b), c) and/or d).

4. Vesicle according to any one of the preceding claims, characterised in that the pore unit has an inside pore diameter having a width of more than 1 nm.

5. Vesicle according to any one of the preceding claims, characterised in that the pore unit forms an enantioselective pore.

6. Vesicle according to any one of the preceding claims, characterised in that the vesicle has a positively charged oligomer or polymer in the vesicle interior.

7. Vesicle according to claim 6, characterised in that the vesicle contains polylysin in the vesicle interior.

8. Use of a vesicle according to any one of the preceding claims for binding a substance.

9. Use according to claim 8, wherein the substance to be bound is a nucleic acid.

10. Method of binding a nucleic acid, which comprises bringing the nucleic acid to be bound into contact with a vesicle according to any one of claims 1 to 7.

11. Method of releasing a nucleic acid, which comprises the steps:
a) binding a nucleic acid in a vesicle by a method according to claim 10, and
b) then releasing the bound nucleic acid by applying a shear stress to the vesicle and/or dissolving the vesicle and/or by adding a salt.


SEQUENCE LISTING (2 sequences)

SEQ ID NO: 1 - DNA, 39 bp, Artificial Sequence
  forward primer for PCR amplification
atgggtctcgaattccggatgcgcgctgaaattatgcgt

SEQ ID NO: 2 - DNA, 40 bp, Artificial Sequence
  reverse primer for PCR amplification
catgccatggctcgagttagaaacggaaggttgcggttgc